(19) United States
(12) Patent Application Publication
(10) Pub. No.: US 2012/0080327 A1
(43) Pub. Date: Apr. 5, 2012

(54) METHOD FOR EVALUATING OXIDATION STRESS AND USE OF THE SAME

(76) Inventors: Hiroyuki Honda, Nagoya-shi (JP); Mina Okochi, Nagoya-shi (JP); Yayoi Isaji, Nagoya-shi (JP); Yoji Hamada, Nagoya-shi (JP); Hiroshi Nagasaki, Nagoya-shi (JP)

(21) Appl. No.: 13/377,951

(22) PCT Filed: Jun. 18, 2010
(86) PCT No.: PCT/JP2010/060347

§ 371 Date: Dec. 13, 2011

(30) Foreign Application Priority Data
Jun. 20, 2009 (JP) 2009-147094

(51) Int. Cl. G01N 33/72

(52) U.S. Cl. 205/782

(57) ABSTRACT

Provided is a simple, rapid blood cell analysis method (assay method using erythrocytes) utilizing a novel index. Using a measurement system having a working electrode and a counter electrode, an adhesion level of erythrocyte on the working electrode to which a positive potential, or a current that generates a positive potential, is applied is detected by an electrochemical measurement method. Oxidation stress is evaluated using the detection result.

### TECHNICAL FIELD

The present invention relates to an assay method using erythrocytes. Specifically, the invention relates to a method for evaluating oxidation stress and a use thereof (an assay method of risks of vascular disorder and diseases caused by vascular disorder, and the like). The present application claims priority based on the Japanese Patent Application No. 2009-147094 filed on Jun. 20, 2009, and the content of the patent application is incorporated herein by reference in its entirety.

### BACKGROUND ART

An assay carried out by taking a sample from a human body for the purpose of health administration or discovery of disease risks (easiness of affection, developmental probability, etc.) is desirably performed routinely and continuously, and thus, a method with load of minimal invasiveness (blood collection, urine collection, etc.) is adopted. Particularly, a blood examination is a method of providing a lot of highly credible information and has been widely utilized. However, a current blood examination is mainly a serum analysis, and for blood cells, the number thereof is only calculated except for a specific case. A prior technique relating to an assay method using erythrocytes (Patent Document 1) and a prior technique relating to separation of erythrocytes (Patent Document 2) are shown below.

### PRIOR ART DOCUMENTS

### Patent Documents

Patent Document 1: Japanese Unexamined Patent Application (JP-A) No. 6-281622

Patent Document 2: JP-A No. 2000-171461

### SUMMARY OF THE INVENTION

### Problems to be Solved by the Invention

A serum analysis assuredly provides important information relating to a health condition and presence or absence of affection of specific diseases, and the like. However, there are a case where a serum analysis is not effective and a case where suitable determination cannot be made only by the serum analysis. For example, risks of arteriosclerosis diseases such as cardiac infarction cannot be completely controlled with only administration objects of a cholesterol level, a triglyceride level, a blood sugar level, and the like, which are obtained from a serum analysis. It is considered that the reason thereof is because even though levels of these risk factors are equivalent, diurnal variation and responses of tissues and organs are different in individuals.

Examples of reasons why a blood cell analysis is not routinely performed are: (1) blood cells have difficulty in preservation after collection as compared to serum; (2) there is no method that can measure blood cells with a simple technique; and (3) an effective index to be measured is not clarified.

Thus, an object of the present invention is to provide a simple, rapid blood cell analysis method (assay method utilizing erythrocytes) using a novel index.

### Means to Solve the problem

A human erythrocyte is an anuclear cell having a long life of about 120 days, which occupies about a half volume of the blood and a large amount of erythrocytes can be obtained from a small amount of blood. As known for blood types, erythrocytes have been known to reflect differences among individuals, typically represented by, for example, difference in surface sugar chains, and also reflect presence or absence of stress such as low nutrition, oxidation, and the like. Erythrocytes thoroughly circulate in the blood vessel system in a body, and accumulate stress generated in the living body through a blood vessel and blood since they are anuclear, and it is expected that erythrocytes reflect what kind of damage a human body constituted with cells is actually received. As well as focusing attention on this point, in consideration of the fact that a charge on a cell membrane surface changes due to sialic acid desorption in a sugar chain on a cell surface of an erythrocyte, intensive studies have been made to generate a novel assay method. Specifically, with the expectation that when a carrier that is positively charged (positively charged carrier) is used, oxidation stress of erythrocytes can be simply and rapidly detected, various experiments were carried out. As a result, it was revealed that anadhesive property of erythrocytesto a positively charged carrier is changed according to an amount of oxidation stress and that a difference of adhesion can be detected in a short time. That is, it was clarified that a grade of oxidation stress in erythrocytes can be rapidly evaluated according to the adhesion level of erythrocyte to a positively charged carrier. Furthermore, correlation was recognized between an adhesion level of erythrocyte to a positively charged carrier and a risk factor of vascular disorder or arteriosclerosis according to an assay using an animal model. Such findings mean that when an adhesion level of erythrocyte to a positively charged carrier is examined, disease risks based on oxidation stress, and the like, such as vascular disorder or arteriosclerosis can be determined (in other words, the adhesion level of erythrocyte to a positively charged carrier becomes a novel risk marker). On the other hand, an assay system using the adhesion level of erythrocyte to a positively charged carrier as an index is characterized in that operation is simple, and also rapid detection and examination are possible, and the assay system was considered to be also preferable for screening of medical agents to oxidation stress. Based on the above described findings, the present applicants applied for a patent on the invention relating to the oxidation stress evaluation method, and the like (Japanese Patent Application No. 2007-332529). In addition, a part of the findings were presented in the 60th annual meeting of the Society for Biotechnology, Japan (lecture summary of the 60th annual meeting of the Society for Biotechnology, Japan, issued on Jul. 11, 2008, p. 65), IUMRS-ICA 2008 (IUMRS-ICA summary distributed on Dec. 9, 2008), and the 40th autumn meeting of the Society of Chemical Engineers, Japan (summary of research lecture presentation issued on Aug. 24, 2008).

The present inventors obtained the above described findings and then further promoted studies. The result thereof showed that an electrochemical measurement method is effective for a detection means of an adhesion level of erythrocyte. When an examination in which oxidation stress in a diabetic condition was reproduced was performed, it was shown that a state of oxidation stress load can be simply determined and evaluated according to an electrochemical measurement method. Furthermore, it was confirmed from an experiment using a clinical sample that rapid, simple determination and evaluation are possible according to the measurement method, and the measurement method is useful for determination of disease risks due to oxidation stress.

The present inventions shown below are based on a series of achievements finally obtained from ceaseless studies made by the present inventors.

[1] An oxidation stress evaluation method, detecting a status of adhesion level of erythrocyte on a working electrode to which a positive potential or a current that generates a positive potential is applied, by an electrochemical measurement method using a measurement system having the working electrode and a counter electrode.

[2] The oxidation stress evaluation method according to [1], including the following steps:

(1) a step of applying a positive potential or a current that generates a positive potential to the working electrode;
(2) a step of contacting a sample containing erythrocytes to the working electrode;
(3) a step of measuring an electrical response in the measurement system, wherein a change is observed when erythrocytes adhere to the working electrode; and
(4) a step of determining an adhesion level of erythrocyte in the sample on the working electrode based on the measurement result.

[3] The oxidation stress evaluation method according to [2], wherein the measurement in the step (3) is carried out in the presence of an electron mediator.

[4] The oxidation stress evaluation method according to [3], wherein the measurement in the step (3) is a CV (cyclic voltammetry) measurement.

[5] The oxidation stress evaluation method according to any one of [1] to [4], wherein the measurement system further has a reference electrode.

[6] A risk assay method of a disorder or disease risk caused by oxidation stress, wherein a disease risk is determined based on the evaluation result of the oxidation stress evaluation method according to any one of [1] to [5].

[7] The risk assay method according to [6], wherein the disorder or a disease caused by oxidation stress is vascular disorder, arteriosclerosis or diabetes complication.

[8] The risk assay method according to [6] or [7], wherein a risk is determined in accordance with a criterion that oxidation stress is strong and a disease risk is high when adhesion level of erythrocyte on a working electrode is low.

### BRIEF DESCRIPTION OF THE DRAWINGS

FIG. 1 shows a relationship between the number of erythrocytes and a current value. The left view is a voltammogram obtained from a CV measurement, and the right view is a plot of oxidation peak current values (Iox.) and erythrocyte concentrations (Ht values);

FIG. 2 shows evaluation of a property of erythrocyte adhesion on a positive potential applied electrode. The figure shows a relationship between an applied voltage in a solution with each salt concentration and a current density obtained from the CV measurement;

FIG. 3 shows an optimization test of erythrocyte concentrations. The figure shows a relationship between the erythrocyte concentrations and the current densities obtained from the CV measurement;

FIG. 4 shows evaluation of adhesion levelof erythrocytes to which oxidation stress caused by hyperglycemia is loaded. The left view is a voltammogram when a sample having an erythrocyte concentration of 0.2% was measured, and the right view is a graph showing a relationship between a glucose load level and a current density. Ht 0.2 represents an erythrocyte concentration of 0.2%, and Ht 0.4 represents an erythrocyte concentration of 0.4%;

FIG. 5 shows an example of a sensor chip structure;

FIG. 6 shows an example of a sensor chip structure having a reference electrode;

FIG. 7 shows evaluation of an adhesion level of erythrocytes in blood obtained from a healthy person and a diabetic patient. The figure shows a voltammogram when a sample having a hematocrit concentration of 0.2% was measured;

FIG. 8 shows correlation validation of biochemical analysis values and the result of the erythrocyte adhesion test. The figure shows a plot of current densities and blood sugar levels; and

FIG. 9 shows correlation validation of biochemical analysis values and the result of the erythrocyte adhesion test. The figure shows a plot of current densities and HbAlc.

### DESCRIPTION OF EMBODIMENT

(Oxidation Stress Evaluation Method)

The first aspect of the present invention relates to a method for evaluating oxidation stress (hereinafter also referred to as the “oxidation stress evaluation method”). An oxidation level in a living body is usually kept almost constant, but if the balance between an active oxygen species production system and a reduction system disrupts, “oxidation stress” is generated. The “oxidation stress” can be grasped as an index which expresses a result of accumulating oxidation effects. In the evaluation method of the present invention, an extent of the “oxidation stress” (including presence or absence) is evaluated using erythrocytes obtained from a test subject. In the present invention, damage to an erythrocyte membrane due to oxidation stress is evaluated, using a change in the adhesion level of erythrocyte on a positively charged electrode as an index. More specifically, using a measurement system having a working electrode and a counter electrode, the adhesion levelof erythrocyte on the working electrode to which a positive potential or a current that generates a positive potential is applied is detected by an electrochemical measurement method, and the detection result is used for evaluation of oxidation stress. When the oxidation stress increases, the adhesion levelof erythrocyte on the working electrode lowers, and a shielding effect to the electrode is reduced. As a result, electrical responses in the measurement system such as a current value and an impedance change. In the present invention, utilizing such change in electrical responses, which is caused according to an extent of oxidation stress, oxidation stress is determined and evaluated (the detail is described later).

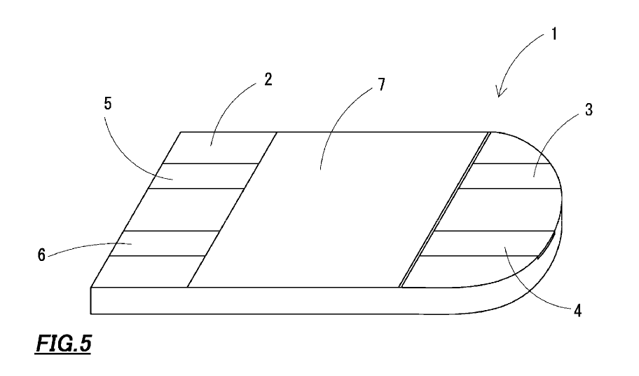

An electrode system having a working electrode and a counter electrode is utilized in various biosensors (for example, JP-A No. 3-202764 and JP-B No. 8-10208). Such known biosensors may be used (see FIG. 5). In addition, a structure of a biosensor and an electrochemical measurement method using the biosensor are described in detail in, for example, Reality of Bioelectrochemistry-Practical Expansion of Biosensor and Biobattery—(issued in March, 2007, CMC Publishing Co., Ltd.).

Materials of each electrode are not particularly limited. Examples of electrode materials of a working electrode and a counter electrode include Au, C, Pt, and Ti.

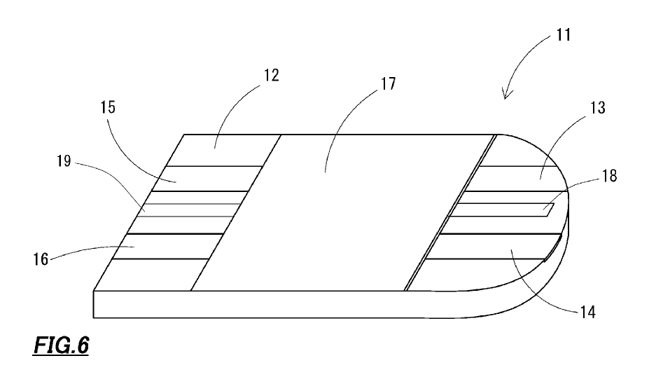

It is preferred that a measurement system also having a reference electrode is used (see FIG. 6). Use of such a measurement system, which is a so-called three-electrode system, makes it possible to express a potential of a working electrode based on a potential of a reference electrode. A measurement system using a constant-potential electrolysis equipment (potentiostat) is more preferably used. When a constant-potential electrolysis equipment is used, a potential of a working electrode can be kept constant and an electrochemical reaction can proceed, and measurement precision is thus improved. Examples of materials of the reference electrode include Ag/AgCl. A saturated calomel electrode can also be adopted as a reference electrode.

Typically, the following steps are carried out:

(1) a step of applying a positive potential or a current that generates a positive potential to a working electrode;
(2) a step of contacting a sample containing erythrocytes to the working electrode;
(3) a step of measuring an electrical response in the measurement system, wherein a change is observed when erythrocytes adhere to the working electrode; and
(4) a step of determining an adhesion level of erythrocyte in the sample on the working electrode based on the measurement result. Each step will be explained below.

(1) Potential Application Step

In this step, a positive potential or a current that generates a positive potential is applied to a working electrode. In other words, a potential is applied to a working electrode so that a positive current is generated in a measurement system. Thereby, the working electrode has positive charge, which enables adhesion of erythrocytes to the working electrode.

Typically, a voltage is applied in a state of impregnating an electrode into a solution containing an electrolyte, and positive charge is given to the working electrode. Various salts can be used as the electrolyte. Examples of the electrolyte include NaCl, KCl, and CaCl2. A suitable electrolyte concentration can be easily set in a preliminary experiment by a person skilled in the art. The suitable electrolyte concentration varies depending on a structure of a measurement system, and an example of a concentration when an NaCl solution is used is shown to be 5 mM to 100 mM. In addition, a buffer solution such as PBS (phosphate-buffered saline) may be also adopted as a solution herein.

A necessary voltage may be set in consideration of a structure of a measurement system (a structure of an electrode, an electrolyte concentration, etc.). A suitable voltage can be easily set in a preliminary experiment by a person skilled in the art. The suitable electrolyte concentration varies depending on a structure of a measurement system, and an example of the voltage is shown to be 0.5 V to 1.0 V. An application time of a potential is not particularly limited. As shown in examples described later, it has been confirmed that the object can also be achieved with a short-time application. In addition, an application time of a potential is preferably short in order to carry out the evaluation rapidly. Therefore, the application time of a potential is set to, for example, 2 seconds to 2 minutes.

(2) Contact Step

In this step, a sample and a working electrode are contacted. A sample containing erythrocytes is used. A kind of a sample is not particularly limited as long as the sample contains erythrocytes. For example, venous blood, whole blood derived from capillary blood vessel, an erythrocyte fraction, erythrocytes (erythrocyte suspension) separated and purified from whole blood, an erythrocyte fraction, or the like can be used as a sample. A preparation of a sample may be followed by a general method. When an erythrocyte concentration is too high, anadhesive property of erythrocytes on a working electrode is not observed, and thus, a sample having an erythrocyte concentration adjusted to, for example, 0.05 to 1% (v/v), preferably 0.1 to 0.4% (v/v), is used. A suitable erythrocyte concentration can be easily set in a preliminary experiment by a person skilled in the art. As a diluting solution used for adjustment of the erythrocyte concentration, various salt solutions (NaCl solution, KCl solution, etc.) and PBS, and the like can be exemplified. In addition, a sample containing an erythrocyte membrane ghost as erythrocytes may be also used.

In order to bring a sample into contact with a working electrode, for example, the working electrode may be immersed into the sample. When a sensor having a working electrode and a counter electrode formed on a substrate (see FIG. 5, similar sensors are also disclosed in JP-A No. 3-202764 and JP-B No. 8-10208) is used, for example, a sample may be dropped or applied on the formed region of the working electrode and the counter electrode.

The contact time of a sample and a working electrode may be suitably set in consideration of an electrode system, a measurement system, the number of erythrocytes in the sample, etc. An example of the contact time is shown to be 10 seconds to 5 minutes. From the viewpoint of rapidly obtaining evaluation results, the contact time is preferably short. As shown in examples described later, even when the contact time was about 1 minute, a difference of the adhesion level could be detected.

This contact step may be carried out at the same time with the above described potential application step. That is, a potential may be applied in a state of contacting the sample and the working electrode. For example, application of a potential may be initiated at the same time of starting contact of the sample and the working electrode. Alternatively, after the sample and the electrode are contacted, a potential may be applied while keeping the contact state.

(3) Electrical response measurement step

An electrical response in a measurement system wherein a change is observed when erythrocytes adhere to a working electrode is measured in this step. The “electrical response” herein is not particularly limited as long as a change is recognized when erythrocytes adhere to a working electrode. For example, for “a measurement of an electrical response”, a CV (cyclic voltammetry) measurement, a measurement of a resistance value, a measurement of an impedance value, or a CP (chronopotentiometry) measurement is performed. Preferably, a CV measurement is carried out. This is because a shielding effect of an electrode due to erythrocyte adhesion can be measured with high sensitivity from a change in current values in an oxidation and reduction potential of an electron mediator and stable data can be obtained.

For example, a solution containing an electrolyte is prepared and in a state of impregnating an electrode into the solution, an electrical response is measured (the first embodiment). The electrode may be washed before the measurement to remove erythrocytes that do not adhere to the electrode. For example, a working electrode after the contact step is immersed into a washing liquid for a predetermined time and unnecessary erythrocytes are washed to be removed. A buffer solution such as saline or PBS may be used for washing. The washing operation may be also carried out plural times (for example, 2 to 5 times). Preferably, a washing liquid having a salt concentration at the same level as a sample used in the contact step is used so as not to damage erythrocytes adhered to a working electrode.

An electrical response may be also measured while maintaining a state of contacting a sample and an electrode after the contact step (the second embodiment). In the case of this embodiment, a power distribution state necessary for the measurement of an electrical response is formed through a liquid component in the sample. Therefore, the contact step and the electrical response measurement step can be sequentially carried out.

The electrical response measurement step is preferably carried out in the presence of an electron mediator. This is because a measurement with high sensitivity and high precision becomes possible. For example, an electrolyte containing an electron mediator may be used in the case of the above described first embodiment. On the other hand, in the case of the above described second embodiment, for example, an electron mediator may be added to a sample before or after the contact step. For the electron mediator, metal complexes or organic compounds such as potassium ferricyanide, ferrocene derivates, and quinone derivatives can be used.

Herein, when a sensor having a layer containing an electron mediator, formed on a working electrode and a counter electrode (hereinafter referred to as a “reaction layer”) is utilized, addition of the electron mediator can be omitted. In this case, the sample is introduced into the reaction layer, thereby causing contact of the sample and the electrode. Subsequently, a power distribution state is formed by a liquid component in the sample and the electron mediator, which enables a measurement of an electrical response.

(4) Determination Step

Then, the adhesion level of erythrocyte on a working electrode in a sample (erythrocyte deposition extent) is determined based on the measurement result. For example, the adhesion level of erythrocyte can be determined by comparison between the measurement result of a control and the measurement result of the sample. A graph, a table, or the like, which expresses a correspondence relationship between the adhesion level of erythrocyte and the measurement results is formed and compared to the measurement results of the sample, and thus, the adhesion level of erythrocyte may be also determined.

When a CV (cyclic voltammetry) measurement is carried out as the “measurement of electrical responses”, for example, oxidation stress is determined and evaluated based on a current density of an oxidation-reduction peak. When the oxidation stress is large, a current density increases, and when the oxidation stress is small, the current density decreases. Therefore, oxidation stress can be determined and evaluated using the degree of the current density as an index.

(Risk Assay Method)

The second aspect of the present invention relates to an assay method of a disorder or disease risk caused by oxidation stress. The “disease risk” in the present specification is a term which inclusively expresses a possibility of being affected with disorders or diseases and possibility of developing (worsening) disorders or diseases. Therefore, information (grade of disease risk) obtained by performing the disease risk assay method of the present invention becomes an index for evaluating a possibility of being affected with disorders or diseases, and also in the case where a person (patient) who is affected with a disorder or a disease is a test subject, such information becomes a useful index for evaluating whether the disorder or disease tends to develop (worsen) or tends to improve (alternatively in remission). Accordingly, the disease risk assay method of the present invention provides useful information which contributes to inhibition of affection or development, delay of development or progress (worsening), and improvement of quality of a patient's life (QOL: Quality of Life). In addition, a test subject is not particularly limited. That is, the present invention can be widely applied to those who require risk determination of disorders or diseases caused by oxidation stress.

In the disease risk assay method of the present invention, the “disease risk” relating to disorders or diseases caused by oxidation stress is examined. The oxidation stress has been known to be factors of development or progress of various disorders and diseases. The disease risk assay method of the present invention can be widely applied to disorders or diseases caused by oxidation stress. The wording of “caused by oxidation stress” means that oxidation stress is directly or indirectly a development factor or progress factor. Examples of the “disorders or diseases caused by oxidation stress” include vascular disorder, arteriosclerosis, diabetes complication, metabolic syndrome, cancer, and Alzheimer's disease. Preferably, a risk of vascular disorder, arteriosclerosis or diabetes complication is an object to be tested.

In the disease risk assay method of the present invention, a risk is determined based on the evaluation results obtained by the above described oxidation stress evaluation method. For example, a disease risk can be determined according to the determination criterion such that “when an adhesion level of erythrocyte on a working electrode is low, oxidation stress is strong, and the disease risk is high.”

The above described oxidation stress evaluation method is carried out with time (preferably periodically) to examine variation in oxidation stress amounts, thereby making it possible to monitor disease risks.

### EXAMPLES

<Relationship Between the Erythrocyte Number and a Current Value>

1. Method

(1) Finger prick blood was taken from a healthy adult and an erythrocyte suspension having each erythrocyte concentration was prepared using a saline (sample).

(2) The sample (10 μl) was put on a sensor chip having an electrode system (a working electrode, a counter electrode and a reference electrode) (DEP Chip SP-N manufactured by Bio Device Technology Co., Ltd.: a working electrode and a counter electrode are made from carbon, a reference electrode is made from Ag/AgCl) formed on a substrate and incubated for 5 minutes.

(3) A saline (10 μl) containing 8 mM of potassium ferricyanide (FCN) was added and a CV measurement (cyclic voltammetry) was performed. Hereinafter, the electrochemical measurement system HZ-5000 manufactured by HOKUTO DENKO CORPORATION was used in all electrochemical measurement experiments.

2. Results

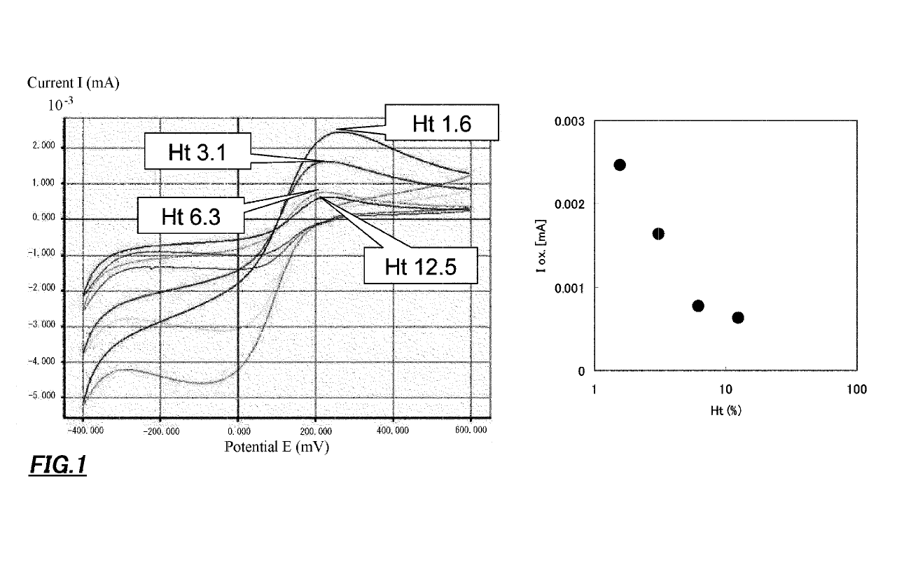

The measurement results are shown in FIG. 1. A voltammogram in which an oxidation-reduction peak decreases along with increase of an Ht value since erythrocytes shield an electrode was obtained (the left view in FIG. 1). A plot of the oxidation peak current values and the Ht values was shown in the right column of FIG. 1. It was confirmed that the number of erythrocytes on the electrode can be precisely presumed by a CV measurement in the presence of a mediator when erythrocytes were within the range of not completely covering the electrode.

<Evaluation of Adhesion Property of Erythrocyte on Positive Potential Applied Electrode>

1. Method

(1) Finger prick blood was taken from a healthy adult and an erythrocyte suspension having an erythrocyte concentration of 0.5% (v/v) (described as Ht 0.5%) was prepared using a saline (sample).

(2) A constant potential (0.7, 0.8, 0.9 V vs. Ag/AgCl) was applied to a working electrode in a sensor chip (DEP Chip SR-N manufactured by Bio Device Technology Co., Ltd.: a working electrode is made from gold, a counter electrode is made from carbon, and a reference electrode is made from Ag/AgCl) in an NaCl solution having each concentration (0, 6, 30, 150 mM) for 5 seconds, and the working electrode was then immersed into the sample and incubated for 1 minute, thereafter washing with a saline.

(3) The sensor chip was immersed into a saline containing 10 mM of potassium ferricyanide and 10 mM of potassium ferrocyanide and a CV measurement (cyclic voltammetry) was performed to analyze a current density of an oxidation-reduction peak.

2. Results

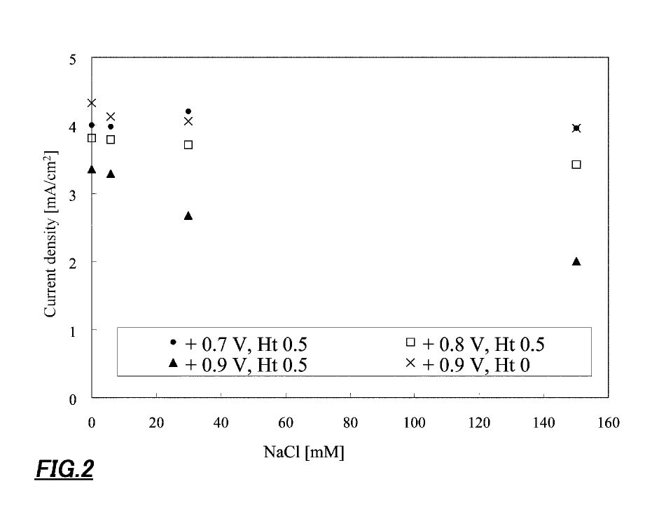

As shown in FIG. 2, change in the current density was not observed at 0.7 V, but the current densities decreased at 0.8 V and 0.9 V applications. In addition, since in the case of applying 0.9 V and not contacting with erythrocytes, decrease in the current density was not occurred, decrease in the current densities due to potential applications of 0.8 V and 0.9 V was caused by adhesion (deposition) of erythrocytes. When an NaCl concentration at potential application is high, an electrical double layer becomes thin, a gap of the applied potential loads on a shorter distance, and an electrical energy on the electrode surface increases, and thus, as a salt concentration is higher, a current density decreases (adhesion of erythrocytes increases). However, electrolysis of a solution easily occurs at a higher salt concentration and a reaction on the electrode surface becomes more complicated, and thus, decrease in the current density due to erythrocyte adhesion is observed and it is necessary that a salt concentration and an application voltage are set within the range where electrolysis of the solution is not generated. For example, when 0.9 V was continuously applied in 150 mM of NaCl in the case of the sensor chip in this time, an electrolytic current is observed, and therefore, it is considered that 0.9 V application in 30 mM of NaCl is suitable. Accordingly, it was confirmed that erythrocytes adhered to the electrode to which a positive potential was applied in a solution having a suitable concentration, and a current density decreased.

<Optimization of Erythrocyte Adhesion Test on Positive Potential Applied Electrode>

1. Method

(1) Finger prick blood was taken from a healthy adult and an erythrocyte suspension having each erythrocyte concentration was prepared using a saline (sample).

(2) A potential of 0.9 V was applied to a working electrode in a sensor chip (the same one as used in the above descried experiment) in an NaCl solution having a concentration of 30 mM for 5 seconds, and the working electrode was then immersed into the sample and incubated for 1 minute, thereafter washing with a saline.

(3) The sensor chip was immersed into a saline containing 10 mM of potassium ferricyanide and 10 mM of potassium ferrocyanide and a CV measurement (cyclic voltammetry) was performed to analyze a current density of an oxidation-reduction peak.

2. Results

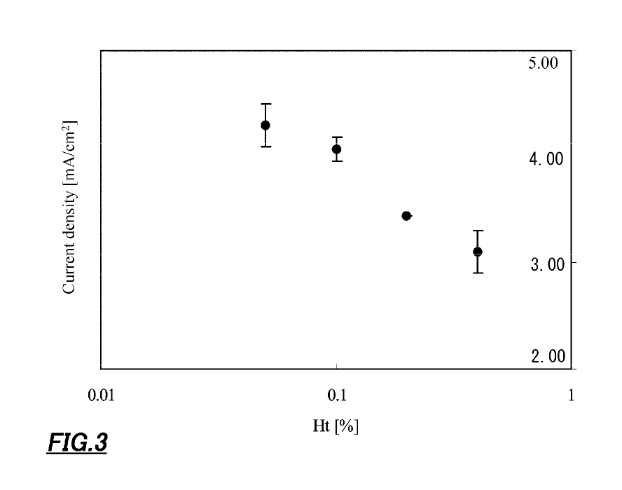

As shown in FIG. 3, when a sample from a healthy subject was used, signals corresponding to the number of erythrocytes within the range of an erythrocyte concentration from 0.1% (Ht 0.1) to 0.4% (Ht 0.4) were detected. In the erythrocyte concentration of 0.05% (Ht 0.05), a value approximately equivalent to the current density in the case of no erythrocyte was obtained. In order to perceptively detect lowering of the current density due to decrease of erythrocyte adhesion, it is necessary that a sample concentration is set within the range where a current value responds the most to decrease of the deposition number. For example, when the sensor chip applied at 0.9 V vs. Ag/AgCl for 5 seconds in 30 mM of NaCl in this time is used, it can be considered that measuring a sample having an erythrocyte concentration of 0.1% to 0.4% is suitable. Accordingly, it was confirmed that a change in the number of erythrocyte which adhere to a positive potential applied electrode can be detected from the change in the current density if having a suitable sample concentration.

<Evaluation of Adhesion Level of Erythrocytes Loaded with Oxidation Stress Caused By Hyperglycemia>

1. Method

(1) Venous blood was taken from a healthy adult and an erythrocyte suspension having an erythrocyte concentration of 50% was prepared using a saline.

(2) The erythrocyte suspension was dividedly charged into a microtube in an amount of 0.5 ml in each time, added with 0.125 ml of a glucose solution to have final concentrations of 5, 45, and 60 mM, and incubated (37° C., 24 hours).

(3) A solution (0.5 ml) which was washed after loading glucose and adjusted to have an erythrocyte concentration of 40% once again was subjected to a measurement of a lipid peroxide amount using a TBA reaction product as an index, and the remaining solutions were adjusted to have erythrocyte concentrations of 0.2% (Ht 0.2) and 0.4% (Ht 0.4) (samples) and an electrochemical measurement was carried out.

(4) A potential of 0.9 V vs. Ag/AgCl was applied to a working electrode in a sensor chip (the same one as used in the above descried experiment) in 30 mM of NaCl for 5 seconds, and the working electrode was then immersed into a sample and incubated for 1 minute, thereafter washing with a saline.

(5) A sensor chip was immersed into a saline containing 10 mM of potassium ferricyanide and 10 mM of potassium ferrocyanide and a CV measurement (cyclic voltammetry) was performed to analyze a current density of an oxidation-reduction peak.

2. Results

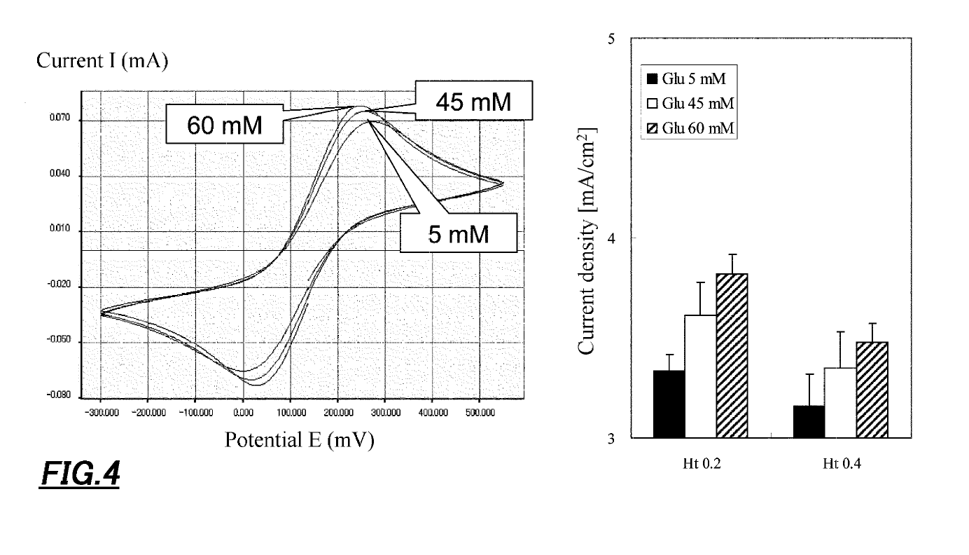

As a result of performing 24-hour glucose load, a lipid peroxide (MDA) amount quantitated by TBA reactivity was 7.3±0.9 nmol/ml-RBC in 5 mM load that is equivalent to a normal blood sugar level; on the other hand, a lipid peroxide amount was increased to 8.9±1.8 nmol/ml-RBC in 45 mM load that is a model of hyperglycemia, and a lipid peroxide amount was increased to 10.7±0.8 nmol/ml-RBC in 60 mM load, and an erythrocyte sample in which oxidation stress due to hyperglycemia was accumulated was obtained. A voltammogram was changed along with increase in a glucose concentration (along with increase in oxidation stress). A voltammogram in the case of measuring the sample having an erythrocyte concentration of 0.2% (Ht 0.2) is shown in the left view of FIG. 4. A current density obtained from this measurement result increased in a hyperglycemia group, and, decrease in the number of deposition of oxidation stress erythrocytes on the positive potential applied electrode could be detected (right view in FIG. 4). Accordingly, it was indicated that a state of oxidation stress load in diabetic condition can be simply determined and evaluated by an electrochemical measurement using a positive potential applied electrode. In addition, it was measureable with 10 μl of a sample with Ht 0.2, which thus theoretically results in being measurable with an ultratrace amount such as 50 nl of blood.

<Verification of Simple Measurement of Clinical Sample>

1. Method

(1) The whole blood taken from a healthy adult and a diabetic patient was diluted by 200 fold using a saline to prepare samples (Ht about 0.2%). Simultaneously, the patient's blood was subjected to a biochemical analysis.

(2) A potential of 0.9 V was applied to a working electrode in a sensor chip (the same one as used in the above descried experiment) in 30 mM of NaCl for 5 seconds, and the working electrode was then immersed into the sample and incubated for 30 seconds, thereafter washing with a saline.

(3) A sensor chip was immersed into a saline containing 10 mM of potassium ferricyanide and 10 mM of potassium ferrocyanide and a CV measurement (cyclic voltammetry) was performed to analyze a current density of an oxidation-reduction peak.

2. Results

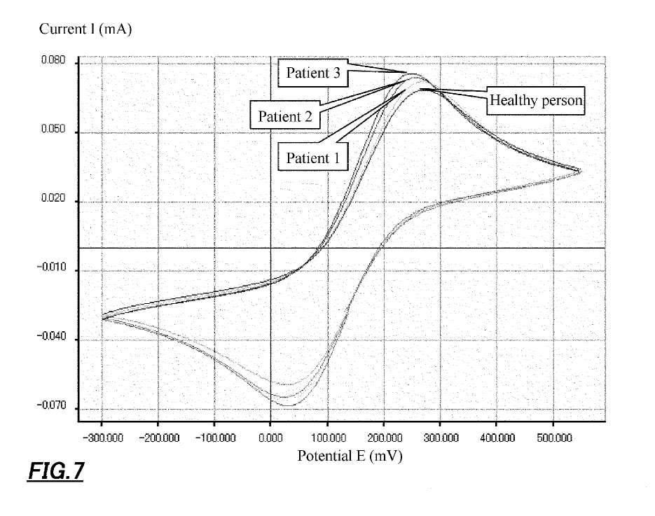

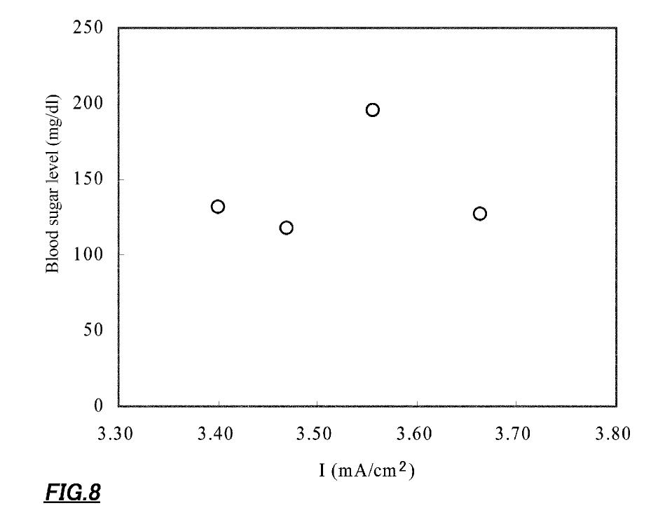

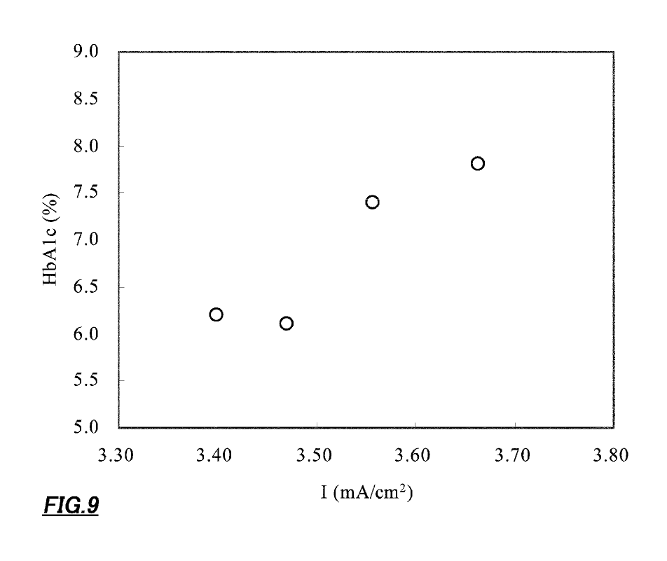

As compared to a healthy person, rise in a current value (decrease in the number of adherent erythrocytes) was confirmed in the patient's blood, and a simplified examination using the whole blood was able to be performed (FIG. 7). Also, there was a tendency such that a current density was not directly correlated to a blood sugar level (FIG. 8), but correlated more to HbAlc reflecting long-term blood sugar control which has a deep relationship with a diabetes complication development risk (FIG. 9). As described above, according to an electrochemical measurement using a positive potential applied electrode, a clinical sample was able to be evaluated for an extremely short time (for a few minutes in this condition) without need of complicated sample preparation and reagent preparation. Oxidation stress derived from a hyperglycemia condition and lipid metabolism abnormality in diabetes becomes a predisposing factor of various diseases typically including arteriosclerosis. This method is the first method of simply measuring a state of mid-and-long-term oxidation stress. In studies in this time, the results of the method showed a positive correlation with HbAlc. The fact supports usefulness of the method which can more simply predict a risk due to oxidation stress.

### INDUSTRIAL APPLICABILITY

According to the present invention, oxidation stress accumulated on an erythrocyte membrane can be rapidly detected and evaluated. The invention also makes it possible to minimally invasively, and rapidly examine risks of disorders or diseases due to oxidation stress. The risk assay method of the present invention can be a useful tool for evaluation of a malignancy grade or a progress grade of various diseases associated with oxidation stress and evaluation of therapeutic effectiveness.

The present invention is not limited to the description of the above embodiments and examples of the invention at all. Various modified embodiments can be included within the range where a skilled person can easily conceived of, without deviating from the description of the scope of patent claims.

Contents of the treatises, published patent bulletins and patent bulletins specified in the present specification are incorporated herein by reference in their entirety.

### EXPLANATION OF SYMBOLS

1, 11 Sensor chip
2, 12 Substrate
3, 13 Working electrode
4, 14 Counter electrode
5, 6, 15, 16, 19 lead
7, 17 Insulating layer
18 Reference electrode

1. An oxidation stress evaluation method, detecting a status of adhesion level of erythrocyte on a working electrode to which a positive potential or a current that generates a positive potential is applied, by an electrochemical measurement method using a measurement system comprising the working electrode and a counter electrode.

2. The oxidation stress evaluation method according to claim 1, comprising the following steps:
(1) a step of applying a positive potential or a current that generates a positive potential to the working electrode;
(2) a step of contacting a sample including erythrocytes to the working electrode;
(3) a step of measuring an electrical response in the measurement system, wherein a change is observed when erythrocytes adhere to the working electrode; and
(4) a step of determining a adhesion level of erythrocyte in the sample on the working electrode based on the measurement result.

3. The oxidation stress evaluation method according to claim 2, wherein the measurement in the step (3) is carried out in the presence of an electron mediator.

4. The oxidation stress evaluation method according to claim 3, wherein the measurement in the step (3) is a CV (cyclic voltammetry) measurement.

5. The oxidation stress evaluation method according to claim 1, wherein the measurement system further comprises a reference electrode.

6. A risk assay method of a disorder or disease risk caused by oxidation stress, wherein a disease risk is determined based on the evaluation result of the oxidation stress evaluation method according to claim 1.

7. The risk assay method according to claim 6, wherein the disorder or disease caused by oxidation stress is vascular disorder, arteriosclerosis or diabetes complication.

8. The risk assay method according to claim 6, wherein a risk is determined in accordance with a criterion that oxidation stress is strong and a disease risk is high when an adhesion level of erythrocyte on a working electrode is low.
